(19) United States
(12) Patent Application Publication
(10) Pub. No.: US 2005/0234492 A1
(43) Pub. Date: Oct. 20, 2005

(54) Lancet device and method of use

(76) Inventors: Fu Hsiung Tsai, San Diego, CA (US); Jinn-nan Lin, San Diego, CA (US); Chia-Lin Wang, San Diego, CA (US)

Correspondence Address: AZURE INSTITUTE;INTELLECTUAL PROPERTY DEPT., 4108 SORRENTO VALLEY BOULEVARD, SAN DIEGO, CA, 92124, US

(21) Appl. No.: 11/081,791

(22) Filed: Mar. 15, 2005

Related U.S. Application Data
(60) Provisional application No. 60/553,223, filed on Mar. 15, 2004.

(51) Int. Cl. A61B 17/32

(52) U.S. Cl. 606/181

(57) ABSTRACT

The present invention is directed to spring-activated devices and methods for lancing the skin to obtain a small amount of blood for metabolic testing. In one embodiment, the depth penetration of the lancet needle can be seen by the patient during lancing the skin. In another embodiment, the device has only five moving parts for cocking and firing, a plunger, two springs, a slider and a button. In order to reduce manufacturing costs, the number of parts used in the device has been reduced and their configuration has been simplified to reduce construction time, and thereby save on labor costs.

This application claims priority to U.S. provisional application Ser. No. 60/553,223, filed Mar. 15, 2004, which is incorporated by reference herein, including all Tables, Figures and claims.

### FIELD OF THE INVENTION

The present invention is directed to lancing devices and methods for use with lancets, for the self-removal of small amounts of body fluid by penetrating a body tissue at a sampling site.

### BACKGROUND OF THE INVENTION

The following Background of the Invention is intended to aid the reader in understanding the invention and is not admitted to be prior art.

The invention relates to lancet devices for withdrawing blood for diagnostic purposes. The self-removal of small amounts of blood from a finger, forearm or thigh for testing purposes is an often daily necessity among patients having metabolic disorders. This practice enables patients, such as diabetics, to monitor specific analytical values in their blood on a regular basis.

Lancets commonly have a metal needle with one end ground to a point. The posterior part of the lancet needle at the opposite end from the tip is commonly enclosed in a lancet body made of a plastic material (e.g., U.S. Pat. No. 3,358,689). During manufacture the lancet needle is usually positioned in a plastic injection mold and the lancet body injected onto it.

Lancing one's own finger is difficult for some due to the pain sometimes associated with the procedure, and the mental stress experienced by some in performing the procedure. A variety of lancet devices have been developed to aid the patient in lancing themselves. In general, these are spring-actuated devices having a housing with a hole at one end. The lancet is secured inside the device and the device is cocked into a “loaded” position. The orifice is placed against the selected puncture site and the device is fired. When the device is fired, the lancet is thrust forward. The needle is protruded from the orifice and pierces the patient's skin. When the procedure is complete, the patient removes the device from the skin and collects a droplet of blood for testing. The testing is commonly conducted with a personal electronic metabolic monitor, such as those that use either electrochemical or optical test strips.

### SUMMARY OF THE INVENTION

The present invention is directed to devices for lancing the skin at a selected body site, in order to withdraw a small sample of blood. One embodiment of the present invention is a lancing device that has a housing and an end piece, which has an orifice. A lancet, which has a needle, is secured within the housing and the device is cocked. Next, the end piece is placed against a puncture site and the device is actuated. The lancet needle is rapidly thrust through the orifice and retracted, thus piercing the skin. In one embodiment, there are only five moving pieces used for cocking and firing the device. A spring loaded plunger is disposed within the device, which is cocked using a slider button disposed within the wall of the housing. The plunger has an arming member that engages an arming post disposed within the housing, to hold the plunger in the cocked position. On the plunger is a collar for securely holding the lancet. To fire the device, a release button disposed within the housing wall is depressed. depression of the release button causes the arming member and arming post to disengage and the device to fire. The firing spring thrusts the plunger forward from the retracted (cocked) position during firing, such that a portion of the needle of the lancet protrudes from the orifice. A return spring mounted on the plunger then retracts the needle and returns the plunger to its starting position. The end piece is assembled of a nose and a base. The nose can be rotated about the base, to adjust the depth of needle penetration. The depth adjustment is indicated with markings on the nose and optionally the base. When the device is held in the patient's hand before and during firing, the patient can see the depth setting markings.

In a first aspect, the present invention provides an apparatus for propelling a lancet that can be activated with one hand. The device has a housing, which has proximal and distal portions, interior and exterior surfaces, and an arming post that includes a catch wall. The housing also has one or more alignment guide slots that are disposed on the interior surface, and an orifice at the distal portion. A front piece is removably attached at the distal portion of the housing and has an orifice through which the lancet emerges when the apparatus is activated. The front piece includes a rotatable nose for adjusting the penetration depth of the lancet. A plunger, which has a resting position, a retracted position, and a lancing position, is disposed within the housing. An arming member is integrally and hingeably connected to the plunger and has a step that engages the catch wall of the arming post to hold the plunger in the retracted position. The plunger also has one or more alignment guides that slideably engage the one or more alignment guide slots on the inside wall of the housing. In one embodiment the device is fired by a spring mechanism. The apparatus also has a firing spring for propelling the plunger from the retracted position to the lancing position. The plunger moves parallel to the axis of the housing when propelled. Also attached to the plunger is a return spring for returning the plunger from the lancing position to the resting position. The apparatus also has a depressible release button, which has a portion that engages the arming member and releases the plunger from the retracted position when the release button is depressed. Depressing the release button causes the plunger to move from the retracted position to the lancing position, and the lancet to protrude from the orifice of the front piece. The apparatus also has a slider button that has a portion that engages the plunger, for moving the plunger from the resting position to the retracted position.

In one embodiment of the apparatus, the plunger has a collar for securing the lancet to the plunger. In a further embodiment, the front piece also has a base, which has a hole and is rotatably connected to the nose. The base has base markings for indicating a selected penetration depth of the lancet. The nose is disposed on the base and rotates about the base and has nose markings. The nose markings align with the base markings to indicate the selected penetration depth of the lancet.

In another embodiment, the front piece is removably attached to the housing by a snap-fit attachment. In certain embodiments, the device has five or fewer moving parts. In some embodiments the arming member also has a slide that interacts with the arming post and guides the arming post to the step to secure the plunger in the retracted position. The base markings and nose markings can be disposed on the device to be visible to the user while the apparatus is placed against the skin of the user.

In another aspect, the present invention provides a method of drawing a small blood sample from a subject using a lancing device that can be operated with one hand. The method involves providing an apparatus of the present invention, moving the slider button toward the proximal portion of the housing to engage the step of the arming member with the arming post, and thereby secure the plunger in the retracted position, and depressing the release button to disengage the arming member from the arming post and cause the plunger to be propelled forward to the lancing position and the lancet to pierce the skin of the subject.

In one embodiment, the patient removes the front piece from the housing and secures a lancet to the plunger, and then reattaches the front piece to the housing. This may be followed by a step of rotating the nose to select a penetration depth for the lancet. The method can be carried out with one hand operation.

In another embodiment, when the slider button is moved toward the proximal portion of the housing, the arming member engages the catch wall of the arming post, and the plunger is secured in the retracted position. In still another embodiment, when the plunger is in the retracted position and the release button is depressed, the arming member is freed from the arming post and thereby causes the plunger to be propelled forward and the lancet to protrude from the nose and puncture the skin of the subject.

In a third aspect, the present invention provides an apparatus for propelling a lancet, which can be activated with one hand. In one embodiment, the device has a housing, which has a proximal portion, a distal portion, interior and exterior surfaces, an arming post having a catch wall, one or more alignment guide slots disposed on the interior surface and an orifice at the distal portion. The device also has a front piece that is removably attached at the distal portion of the housing. The front piece has an orifice, through which the lancet emerges when the apparatus is activated, and a means for adjusting the penetration depth of the lancet. A plunger that has a resting position, a retracted position and a lancing position is disposed within the housing. The plunger has a means for arming the plunger, which is integrally and hingeably connected to the plunger. The means for arming the plunger has a means for engaging the catch wall of the arming post and holding the plunger in the retracted position. In certain embodiments, the plunger has a means for aligning the plunger within the housing and for guiding the plunger through the housing as the plunger moves between positions. The plunger also has a means for propelling the plunger from the retracted position to the lancing position, wherein the plunger moves parallel to the axis of the housing when propelled, and a means for returning the plunger from the lancing position to the resting position. In a further embodiment, the device has a means for engaging the arming member and releasing the plunger from the retracted position, and thereby causing the plunger to move from the retracted position to the lancing position, and the lancet to protrude from the orifice of the front piece. In a still further embodiment, the present device has a means for moving the plunger from the resting position to the retracted position. In another embodiment, the plunger also has a collar for securing the lancet to the plunger.

In some embodiments, the front piece also has a base, which has a hole and is rotatably connected to the nose. The base has base markings for indicating a selected penetration depth. In a further embodiment, the means for adjusting the penetration depth of the lance comprises a nose disposed on the base, which rotates about the base. The nose has nose markings that align with the base markings to indicate the selected penetration depth of the lancet.

In still another embodiment, the means for arming the plunger includes a slide, which interacts with the arming post and guides the arming post to a step, which interacts with the arming post to secure the plunger in the retracted position.

In a further embodiment, the base markings and nose markings are disposed on the device to be visible to the user while the apparatus is placed against the skin of the user.

The summary of the invention described above is not limiting and other features and advantages of the invention will be apparent from the following detailed description, as well as from the claims.

### BRIEF DESCRIPTION OF THE DRAWINGS

FIG. 1 provides a perspective view of one embodiment of the present invention, having a housing 110 with proximal 112 and distal 114 ends and an end piece 116.

FIG. 2 provides an exploded view of the device shown in FIG. 1.

FIG. 3 provides a cut-away perspective view of the device shown in FIG. 1. The orientation of the plunger 228, the slider button 236, release button 242, firing spring 234 and return spring 250 within the housing is illustrated.

FIG. 4 is a section view of the device shown in FIG. 1 at A-A, illustrating the orientation of the plunger within the housing and the interaction of the alignment guides 232 with the alignment guide slots 216.

FIG. 5 is a close up of the device shown in FIG. 2, illustrating the arming post 214.

FIG. 6A is a perspective view of one embodiment of the release button 242.

FIG. 6B is a cut-away close-up of the orientation of the release button, the arming post and the arming member 230 when the plunger is in the resting position.

FIG. 7 is a perspective view of one embodiment of the slider button 236.

FIG. 8 is a perspective view of one embodiment of the nose 120.

FIG. 9 is a perspective view of one embodiment of the base 122.

FIG. 10 is an alternative view of the device shown in FIG. 9.

FIG. 11 illustrates the first step in assembling the front piece 116.

FIG. 12 illustrates the second step in assembling the front piece.

FIG. 13 is a perspective view of one embodiment of the base, illustrating an orientation key 220.

FIG. 14 is an alternative perspective view of the device shown in FIG. 13, illustrating the arrangement of the orientation keys and a detent-receiving groove 226.

FIG. 15 illustrates the lock 222, on the exterior of the housing.

FIG. 16 illustrates one embodiment of the plunger showing the attachment of the arming post 230, the firing spring and the off-set parallel axis of the return spring.

FIG. 17 is a close-up cut-away view of the return spring attachment to the plunger and the slider button.

FIG. 18 is a close-up cut-away view of the firing springs attachment to the plunger and its interaction with the proximal, interior surface of the housing.

FIG. 19 is a cutaway view of the device shown in FIG. 1, illustrating the arrangement of elements when the device is in the resting position. Note that neither the firing spring nor the return spring is compressed.

FIG. 20 is a cutaway view of the device shown in FIG. 1, illustrating the arrangement of elements when the device is in the process of being cocked. Note that the return spring has been fully compressed and the firing spring is partially compressed.

FIG. 21 is a cutaway view of the device shown in FIG. 1, illustrating the arrangement of elements when the device is cocked and the plunger has been moved to the retracted position. Note that both the return spring and the firing spring have been fully compressed.

FIG. 22 is a cutaway view of the device shown in FIG. 1, illustrating the arrangement of elements after the device has been cocked and prior to firing. Note that the return spring has moved the slider button to its original position and the firing spring is fully compressed.

FIG. 23 is a cutaway view of the device shown in FIG. 1, illustrating the arrangement of elements after the device has been fired. Note that the stylet protrudes from the orifice of the device, the firing spring has moved forward with the plunger and the return spring is fully compressed. After firing, the device returns to the configuration shown in FIG. 20.

### DETAILED DESCRIPTION

In the following detailed description, reference is made to the accompanying drawings that form a part hereof, and in which is shown by way of illustration specific embodiments in which the invention may be practiced. It is understood that other embodiments may be utilized and structural changes may be made without departing from the scope of the present invention.

The devices and methods of the present invention enable the easy withdrawal of a droplet of blood from a selected puncture site. The devices allow adjustment of puncture depth and the reduction of pain associated with needle penetration. Additionally, the devices provide for reduced manufacture costs associated with the number of parts required to be manufactured and stocked. Labor costs are reduced reducing the amount of time required to assemble each unit. Reduced assembly time is achieved by first, reducing the number of parts to be assembled, and second, simplifying the part arrangement to make it easier to assemble each device.

Lancet Device

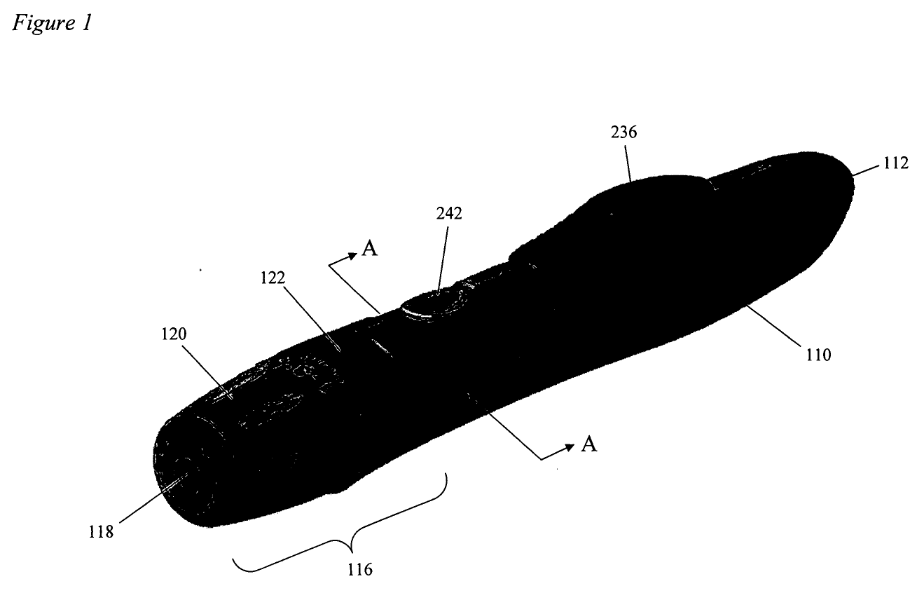

The following discussion relates to those embodiments illustrated in the examples. Of course other embodiments are possible without departing from the scope of this invention. The present invention provides an apparatus for propelling a lancet 246. The apparatus can be activated with one hand. FIGS. 1-3 illustrate one embodiment of the present device, which has five or fewer moving parts required to cock and fire the device. The device has a housing 110, which has a proximal portion 112 and a distal portion 114. The housing also has interior and exterior surfaces (210 and 212, respectively). Disposed on the interior surface of the housing is an arming post 214, having a catch wall 510 (see FIG. 5). One or more alignment guide slots 216 are disposed on the interior surface of the housing. The housing has an orifice 218 at its distal portion. With the exception of the firing 234 and return 236 springs, which will be discussed in greater detail below, all parts of the device are made of injection molded plastic.

In general, a “lancet” for use with a lancing device is a small, disposable device for piercing the skin that has a very short, sharp metal needle and a plastic body. A variety of lancets are currently on the market today. Lancets can be constructed by placing the needle in an injection mould and then filling the mould with plastic.

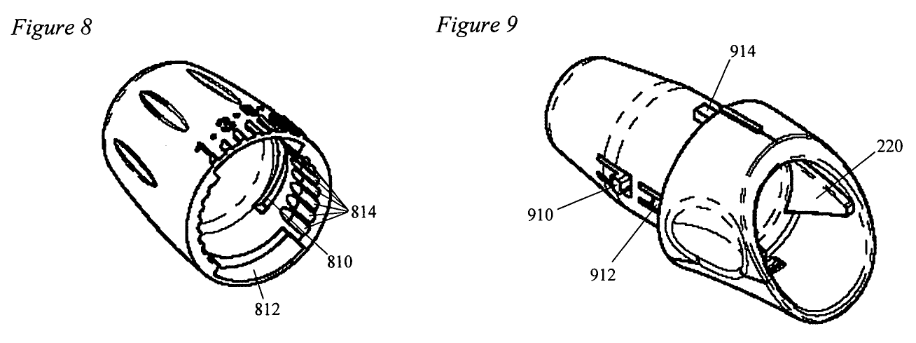

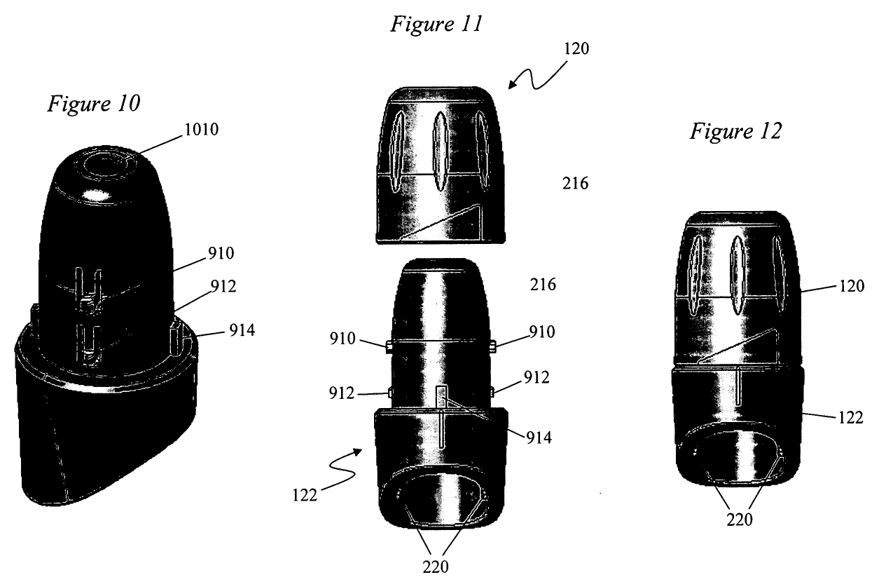

A front piece 116 can be removably attached to the distal portion of the housing. In one embodiment the front piece has an orifice 118 through which the lancet needle emerges when the apparatus is activated. The front piece can be assembled of two parts, a nose 120 and a base piece 122 which snap together during assembly (see FIGS. 8-14). Both the nose and the base have an orifice. When the two pieces are snapped together, the nose orifice (not shown) and the base orifice 1010 align to create the front piece orifice, through which the lancet needle protrudes.

The nose rotates about the base piece, for adjusting the penetration depth of the lancet. As shown in FIG. 8, the interior surface of the nose has a helical groove 810 and an annular groove 812. Also disposed on the interior surface of the nose are a plurality of parallel groves 814 with ridges there between, in alignment with the axis of the device. Referring to FIG. 9, the base has a first detent 910 about which the helical groove slides when the nose is rotated. As the nose is rotated about the base in such a direction that the top of the helical groove moves toward the first detent, the penetration depth is reduced. Conversely, as the nose is rotated about the base in such a direction that the bottom of the helical groove moves toward the first detent, the penetration depth is increased. The depth setting is indicated to the user by the interaction of the parallel grooves 814 on the interior of the nose and a second detent 912 on the base. Additionally, india on the exterior of the nose, corresponding to a location of the first detent along the axis of the helical groove, may indicate the depth setting (see discussion below). The second detent is flexibly disposed on the side of the base. As the nose is rotated about the base, the second detent slides up and over the ridges, and then back into the groves. The second detent can move in and out of the grooves, and over the ridges because it is attached to the base by a flexible arm. Thus, as a ridge makes contact with the second detent, the ridge depresses the second detent. As the ridge moves away from the second detent, the detent returns to its original orientation. As the second detent moves from parallel grove to parallel groove, it makes a click that can be felt or heard by the patient as he adjusts the penetration depth.

The base has a third detent 914 about which the annular groove slides. The interaction of the third detent and the annular groove limits the total amount of the nose about the base.

To assist the patient in accurately selecting a penetration depth, the nose and base may carry indicia that correlate with position of the first detent along the axis of the helical groove. The indicia may take a variety of forms as long as they convey to the patient the increase or decrease of needle penetration associated with a particular nose and base orientation. For example, in FIG. 1, the nose may carry a series of number and the base a fixed line or arrow, or vice versa. The parts can be arranged so that penetration depth increases as the numbers increase, and the selected penetration depth number aligns with the line or arrow on the base. In another embodiment, bars of increasing height (also shown in FIG. 1) or a right-angled triangle can be used to indicate increased penetration depth, instead of numbers.

In certain embodiments, the indicia are disposed on the nose and base such that the depth setting can be seen by the patient when the patient has placed the nose of the device against his skin and is firing the device.

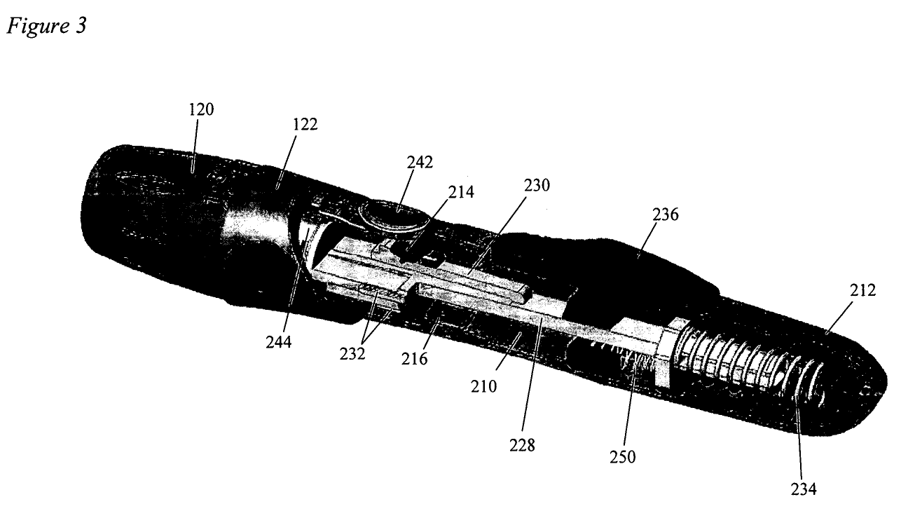

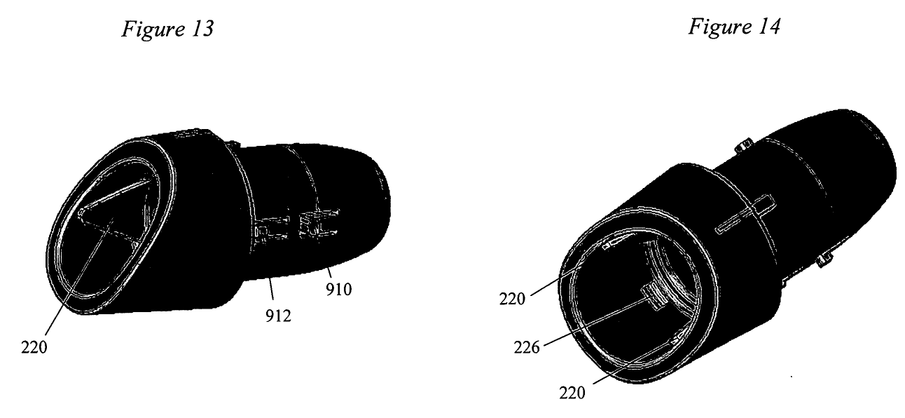

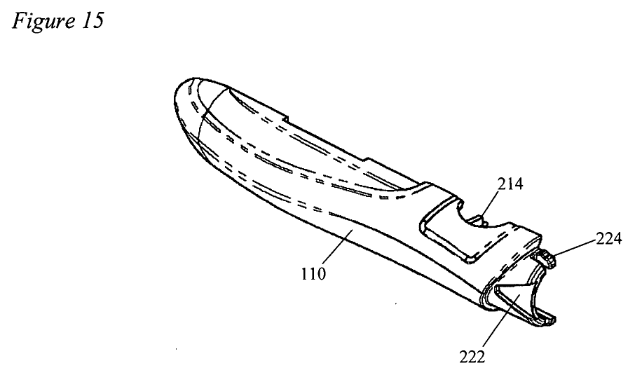

The front piece has a snap-fit attachment to the base. Additionally, the front piece is adapted to fit onto the base in a single orientation. Correct orientation of the end piece is accomplished by the interaction of at least one key 220 on the interior surface of the base and at least one lock 222 on the exterior, distal surface of the housing. The snap-fit attachment is accomplished by mating detents 224 and detent-receiving grooves 226 on the housing and base, respectively (see FIGS. 3 and 13).

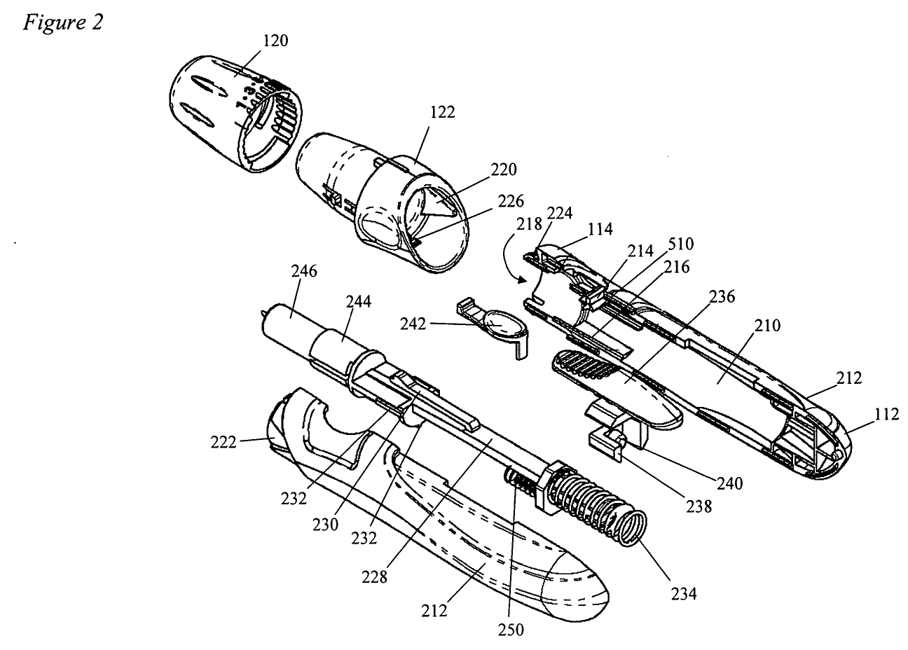

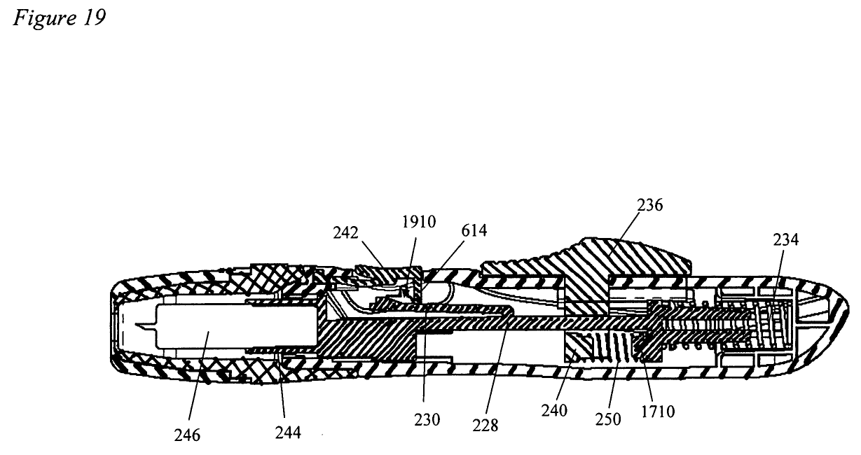

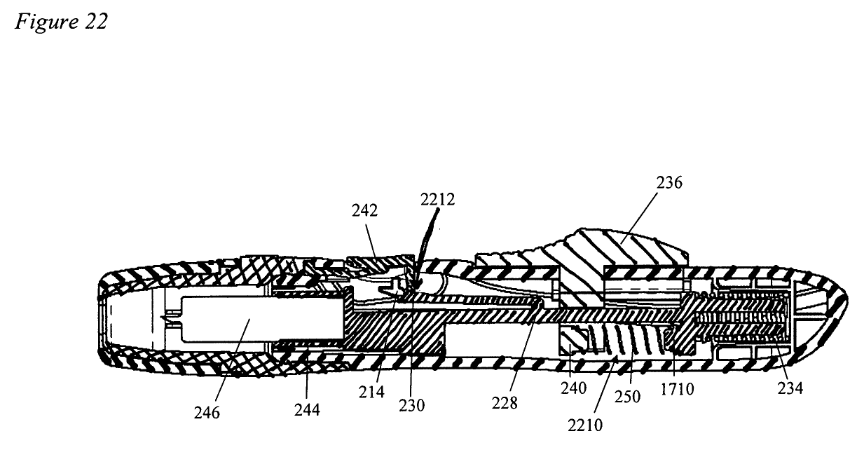

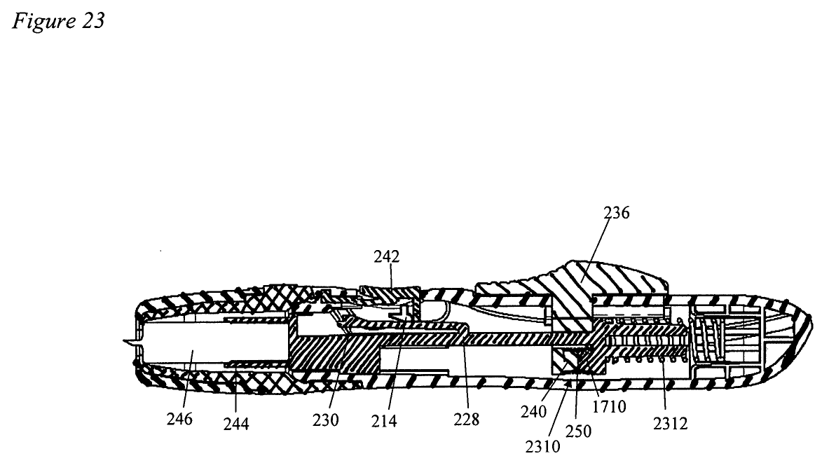

As illustrated in FIGS. 2 and 3, a plunger 228 is disposed within the housing. The plunger has three positions, a resting position (see FIG. 19), a retracted position (see FIG. 22), and a lancing position (see FIG. 23). These positions will be discussed in greater detail below.

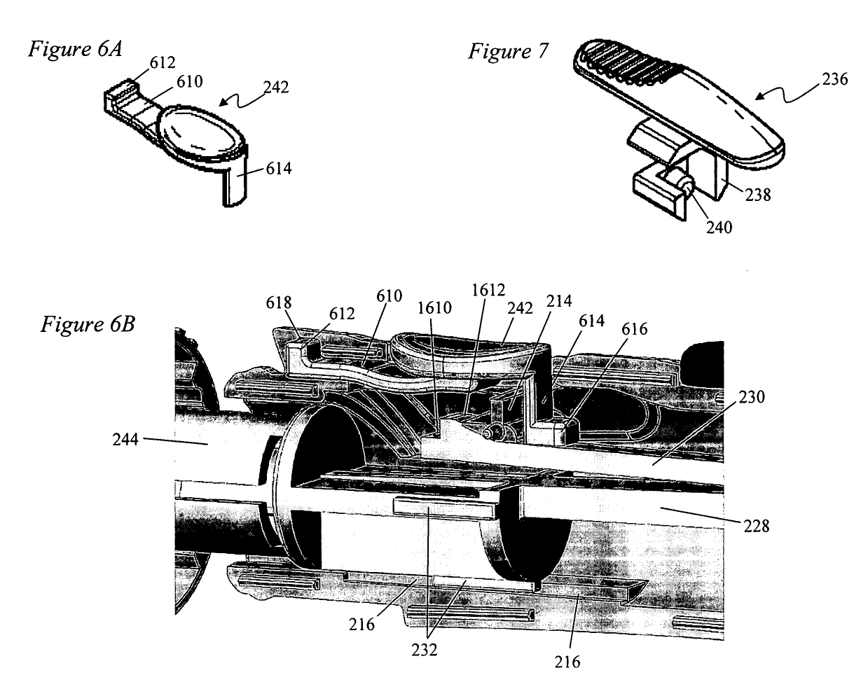

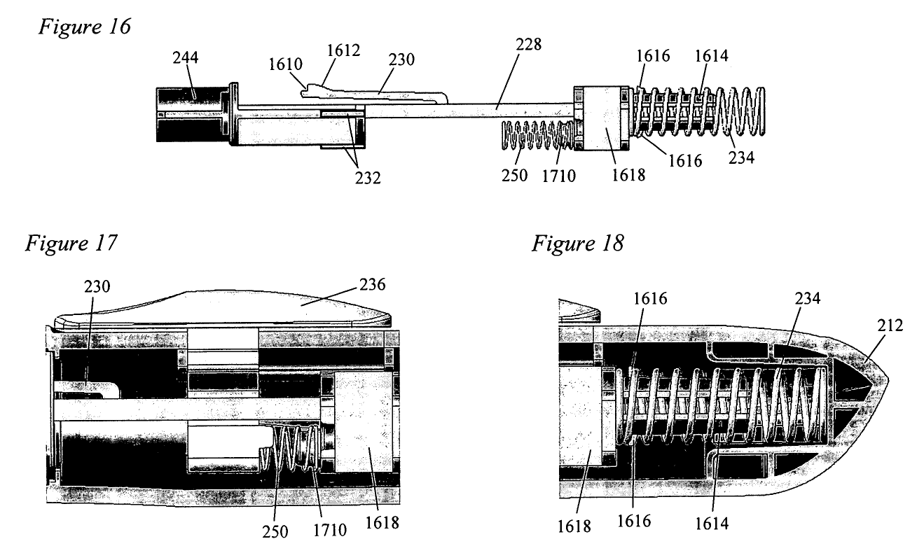

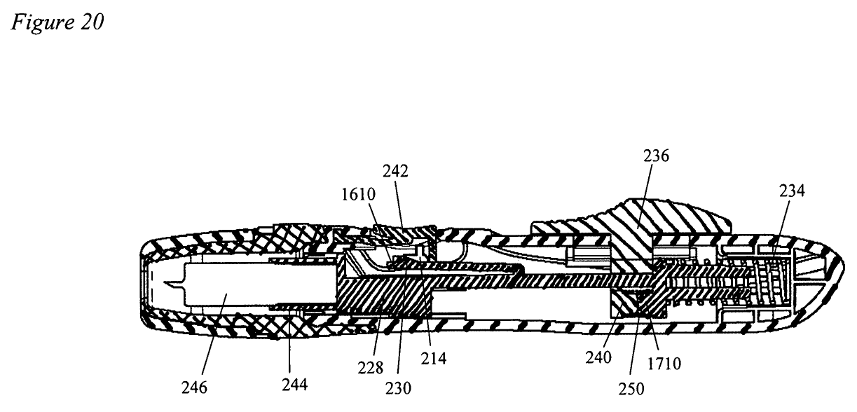

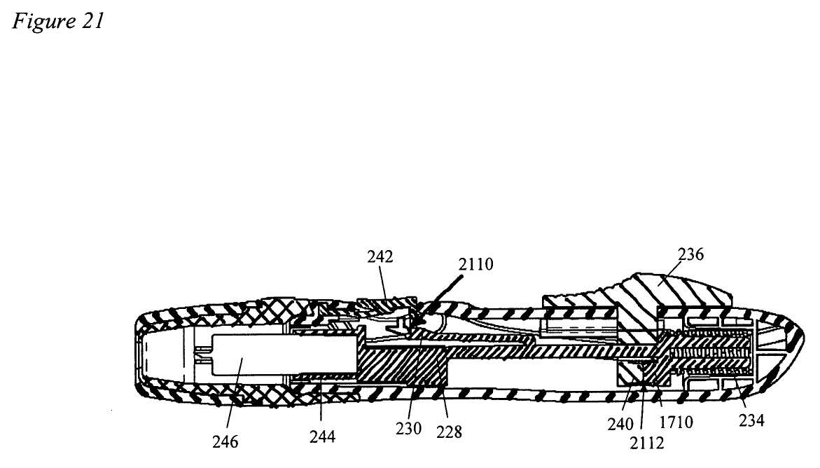

Referring to FIGS. 2, 3, 6B and 16, the plunger has an arming member 230 that is integrally and hingeably connected to the plunger. The plunger and the arming member are injection molded as one piece of substantially rigid plastic. On the end opposite to the connection of the arming member and the plunger, the arming member has a step 1610 (see FIGS. 6B and 16). The step engages the catch wall 510 of the arming post to hold the plunger in the retracted position (see FIG. 21, no. 2110). The arming member also has a slide 1612 (see FIGS. 6B, 16, 19 and 20). As the plunger is moved to the retracted position, the arming post interacts with the slide to press the arming member downward (FIG. 19, no. 1910). The slide guides the arming post to the step to secure the plunger in the retracted position (FIG. 20).

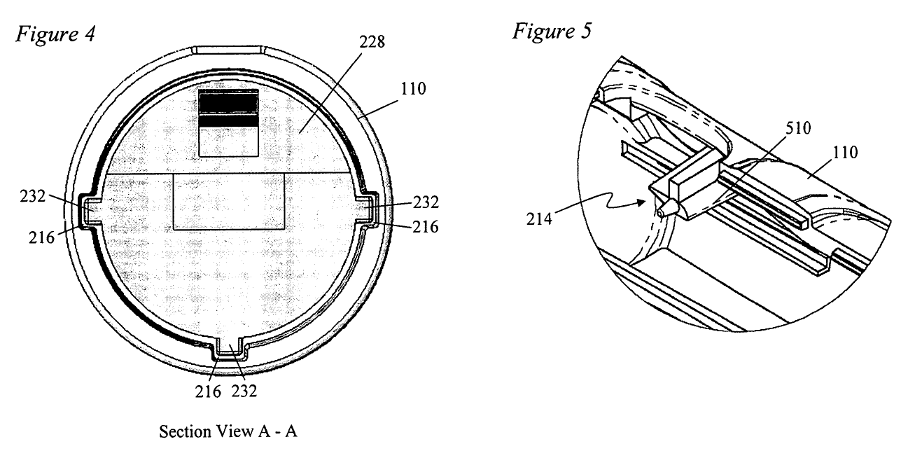

The plunger also has one or more alignment guides 232 that slideably engage the one or more alignment guide slots 216 (see FIGS. 2-4 and 6B). The interaction between the alignment guides with the guide slots is illustrated in cross-section A-A (FIG. 4). The interaction of the alignment guides and the alignment guide slots prevents torque and vibration about the axis of the plunger during operation, to reduce pain to the patient.

As illustrated in the Figures, a firing spring 234, having first and second ends, is slideably attached to the rear end 1614 of the plunger. The first end of the spring may be attached to the plunger by one or more tangs 1616. The plunger may have a compression ring 1618, against which the first end of the spring may press. At its second end, the firing spring presses against the interior proximal end of the housing (see FIG. 18). When the plunger is moved to the retracted position, the firing spring is compressed, causing an increase in its spring energy. When the device is fired, the firing spring releases the stored energy an propels the plunger from the retracted position to the lancing position. As the plunger is thrust forward from the retracted position to the lancing position, it moves parallel to the axis of the housing.

The plunger has a return spring 250 for returning the plunger from the lancing position to the resting position. As illustrated in FIG. 17, the return spring is attached to the plunger by a collar post 1710 on the front of the compression ring. The return spring engages a slider button 236 (see FIGS. 2 and 7). The slider button is slideably disposed in the housing and moves the plunger from the resting position to the retracted position as it is moved. A portion of the slider button, such as a slider collar 238, engages the plunger, for moving the plunger from the resting position to the retracted position. The slider collar wraps around the plunger and on the bottom of the slider collar is a spring post 240. The return spring is attached to the spring post at one end and the collar post at the other end. To cock and arm the device, the patient slides the slider button toward the proximal end of the housing. The slider collar depresses the return spring and pushes the plunger to the retracted position either by pushing in a proximal direction on the collar post or on the compression ring (see FIG. 20). As previously discussed, the step 1610 engages the stop post and holds the plunger in the retracted position. When the device in the retracted position, the return spring is compressed between the collar post and the spring post (see FIG. 21, 2112). After the device is placed into the retracted position, the return spring pushes the slider in a distal direction, to its start position (see FIG. 22, 2210). When the release button is depressed, the plunger is propelled forward by the firing spring, and the return spring becomes compressed between the collar post and the spring post (see FIGS. 23, 2310 and 2314). The return spring moves the plunger back to the resting position, from the lancing position. As the plunger is returned to the resting position, the exposed needle is retracted through the orifice (compare FIGS. 23 and 19).

A depressible release button 242 is mounted in the housing, near the proximal end (see FIGS. 2, 3, 6A and 6B). A cantilevered arm 610 having a detent 612 is integrally attached to the bottom of the release button. The arm and detent fit snuggly into a groove 618 in the housing wall. The groove is adapted to snuggly hold the arm and detent. The release button extends towards the proximal end of the device and over the arming post 214 (see FIG. 6B). The release button also has a release post 614, which optionally has a release detent 616. As illustrated in FIGS. 6B and 19-22, the release post engages the arming member. When the release button is depressed, the release post pushes the arming member downward, such that the step is disengaged from the arming post 2314. As a result, the plunger is released from the retracted position and is thrust forward to the lancing position, by the firing spring 2312. As the plunger arrives in the lancing position, the lancet needle protrudes from the orifice of the front piece and pierces the skin of the patient.

Referring to FIGS. 2, 3 and 6B, the plunger has a collar 244, at its distal end, for holding the lancet 246 in position for lancing the skin. The collar is integrally attached to the plunger because it is injection molded as one piece. The collar is adapted to be slightly flexible such that the body of a lancet can be slid into the collar, but the collar still retains a firm hold on the lancet body.

Another embodiment of the present device is an apparatus for propelling a lancet, which can be activated with one hand and has five or fewer moving parts for cocking and firing the device. In this embodiment, the device has a housing, having a proximal portion, a distal portion, interior and exterior surfaces, an arming post having a catch wall, one or more alignment guide slots disposed on the interior surface and an orifice at the distal portion. A front piece removably attached at the distal portion of the housing. The front piece has an orifice through which the lancet emerges when the apparatus is activated. The front piece also has a means for adjusting the penetration depth of the lancet.

Disposed within the housing is a plunger having a resting position, a retracted position, and a lancing position. The plunger has arming mans, such as a biasing means, which is integrally and hingeably connected to the it. The means for arming the plunger has a means for engaging the catch wall of the arming post and holding the plunger in the retracted position.

The present device has a means for aligning the plunger within the housing and for guiding the plunger through the housing as the plunger moves between positions. The means for aligning and guiding the plunger prevents torque and vibrations when the device is actuated, which would cause increased pain to the patient.

The present device includes a means for moving the plunger from the resting position to the retracted position, to cock the device. After the device has been cocked, it can be fired.

A means for propelling the plunger from the retracted position to the lancing position is attached to the proximal end of the plunger. When the plunger moves from one position to another and when it is propelled, it moves parallel to the axis of the housing. The device also has a means for engaging the arming member and releasing the plunger from the retracted position, and thereby causing the plunger to move from the retracted position to the lancing position. When the plunger moves from the retracted position to the lancing position (i.e., fires), the lancet protrudes from the orifice of the front piece and pierces the patient's skin.

The plunger includes a means for returning the plunger from the lancing position to the resting position. When the plunger returns from the lancing position to the resting position, the lancet is retracted back into the device. If desired, the patient may replace the used lancet with a new one, and re-lance himself.

The lancet is secured to the plunger by a collar integrally attached to the distal end of the plunger. The plunger, the collar and the arming means are injection molded as one piece, of substantially rigid plastic.

In a further embodiment, the front piece has a base that having a hole and is rotatably connected to the nose. The base has base markings for indicating a selected penetration depth. The means for adjusting the penetration depth of the lance comprises a nose disposed on the base that rotates about the base. The nose has nose markings that align with the base markings, to indicate the selected penetration depth of the lancet. These markings can be seen by the patient when they have placed the nose against their skin and are firing the device.

In another embodiment of the present device, the device has five or fewer moving parts for cocking and firing the device. The moving parts may be a plunger, a firing spring, a return spring, a slider button and a release button.

In yet another embodiment, the apparatus has a means for arming the plunger. The means for arming the plunger has a slide, which interacts with the arming post and guides the arming post to a step which interacts with an arming post to secure the plunger in the retracted position.

Methods of Use

The present invention also provides a method of drawing a blood sample from a subject using a lancing device that can be operated with one hand. In one embodiment of the present method, the patient provides the present device in the resting position (FIG. 19) and then cocks the device by moving the slider button toward the proximal portion of the housing. As the patient does this, the step of the arming member engages the arming post, and thereby secure the plunger in the retracted position (see FIG. 21). The patient then places the nose of the device against the selected puncture site and depresses the release button. When the release button is depressed, the arming member is disengaged from the arming post, whereby the plunger is propelled forward to the lancing position and the lancet pierces the skin of the subject (FIG. 23).

In one embodiment, the method includes the step of removing the front piece from the housing and securing a lancet to the plunger, and reattaching the front piece to the housing. The patient may then rotate the nose to select a penetration depth for the lancet.

In certain embodiments of the present method, the steps of cocking and firing the device are carried out with one handed operation.

In another embodiment, the method includes the step of moving the slider button toward the proximal portion of the housing, whereby the arming member engages the catch wall of the arming post, and the plunger is secured in the retracted position. When the plunger is in the retracted position, the release button is depressed, causing the arming member to be freed from the arming post thereby causing the plunger to be propelled forward and the lancet to protrude from the nose and puncture the skin of the subject.

The invention illustratively described herein may be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by various embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

1. An apparatus for propelling a lancet, which can be activated with one hand, comprising:
a housing, having a proximal portion, a distal portion, interior and exterior surfaces, an arming post having a catch wall, one or more alignment guide slots disposed on the interior surface and an orifice at the distal portion;
a front piece removably attached at the distal portion of the housing and having an orifice through which the lancet emerges when the apparatus is activated comprising a rotatable nose for adjusting the penetration depth of the lancet;
a plunger having a resting position, a retracted position, and a lancing position, disposed within the housing and comprising
an arming member integrally and hingeably connected to the plunger and having a step wherein the step engages the catch wall of the arming post to hold the plunger in the retracted position;
one or more alignment guides that slideably engage the one or more alignment guide slots;
a firing spring for propelling the plunger from the retracted position to the lancing position, wherein the plunger moves parallel to the axis of the housing when propelled, and
a return spring for returning the plunger from the lancing position to the resting position;
a depressible release button having a portion that engages the arming member and releases the plunger from the retracted position when the release button is depressed, and thereby causing the plunger to move from the retracted position to the lancing position, and the lancet to protrude from the orifice of the front piece; and
a slider button having a portion that engages the plunger for moving the plunger from the resting position to the retracted position.

2. The apparatus of claim 1 wherein the plunger further comprises a collar for securing the lancet to the plunger.

3. The apparatus of claim 1
wherein the front piece further comprises a base having a hole and being rotatably connected to the nose, and having base markings for indicating a selected penetration depth; and
wherein the nose is disposed on the base and rotates about the base, and having nose markings that align with the base markings to indicate the selected penetration depth of the lancet.

4. The apparatus of claim 3 wherein the front piece is removably attached to the housing by a snap-fit attachment.

5. The apparatus of claim 1 wherein the device has five or fewer moving parts.

6. The apparatus of claim 1 wherein the arming member further comprises a slide which interacts with the arming post and guides the arming post to the step to secure the plunger in the retracted position.

7. The apparatus of claim 3 wherein the base markings and nose markings are disposed on the device to be visible to the user while the apparatus is placed against the skin of the user.

8. A method of drawing a blood sample from a subject using a lancing device that can be operated with one hand, comprising:
providing an apparatus according to claim 1;
moving the slider button toward the proximal portion of the housing to engage the step of the arming member with the arming post, and thereby secure the plunger in the retracted position;
depressing the release button and thereby disengaging the arming member from the arming post, whereby the plunger is propelled forward to the lancing position and the lancet pierces the skin of the subject.

9. The method of claim 8 further comprising removing the front piece from the housing and securing a lancet to the plunger, and reattaching the front piece to the housing.

10. The method of claim 8 further comprising rotating the nose to select a penetration depth for the lancet.

11. The method of claim 8 wherein the method is carried out with one hand operation.

12. The method of claim 8 wherein, when the slider button is moved toward the proximal portion of the housing, the arming member engages the catch wall of the arming post, and the plunger is secured in the retracted position.

13. The method of claim 8 further comprising that, when the plunger is in the retracted position, the release button is depressed, causing the arming member to be freed from the arming post thereby causing the plunger to be propelled forward and the lancet to protrude from the nose and puncture the skin of the subject.

14. An apparatus for propelling a lancet, which can be activated with one hand, comprising:
a housing, having a proximal portion, a distal portion, interior and exterior surfaces, an arming post having a catch wall, one or more alignment guide slots disposed on the interior surface and an orifice at the distal portion;
a front piece removably attached at the distal portion of the housing and having an orifice through which the lancet emerges when the apparatus is activated comprising a means for adjusting the penetration depth of the lancet;
a plunger having a resting position, a retracted position, and a lancing position, disposed within the housing and comprising
a means for arming the plunger integrally and hingeably connected to the plunger and having a means for engaging the catch wall of the arming post and holding the plunger in the retracted position;
a means for aligning the plunger within the housing and for guiding the plunger through the housing as the plunger moves between positions;
a means for propelling the plunger from the retracted position to the lancing position, wherein the plunger moves parallel to the axis of the housing when propelled, and
a means for returning the plunger from the lancing position to the resting position;
a means for engaging the arming member and releasing the plunger from the retracted position, and thereby causing the plunger to move from the retracted position to the lancing position, and the lancet to protrude from the orifice of the front piece; and
a means for moving the plunger from the resting position to the retracted position.

15. The apparatus of claim 14 wherein the plunger further comprises a collar for securing the lancet to the plunger.

16. The apparatus of claim 14
wherein the front piece further comprises a base having a hole and is rotatably connected to the nose, and having base markings for indicating a selected penetration depth; and
wherein the means for adjusting the penetration depth of the lance comprises a nose disposed on the base that rotates about the base, and having nose markings that align with the base markings to indicate the selected penetration depth of the lancet.

17. The apparatus of claim 14 wherein the device has five or fewer moving parts.

18. The apparatus of claim 14 wherein the means for arming the plunger comprises a slide which interacts with the arming post and guides the arming post to a step which interacts with the arming post to secure the plunger in the retracted position.

19. The apparatus of claim 16 wherein the base markings and nose markings are disposed on the device to be visible to the user while the apparatus is placed against the skin of the user.
